(19) United States
(12) Patent Application Publication
(10) Pub. No.: US 2007/0154423 A1
(43) Pub. Date: Jul. 5, 2007

(54) Nail varnish comprising a cellulose ester and short ester solvents in a defined ratio

(75) Inventors: Nathalie Francois, Evry (FR); Roberto Cavazzuti, Paris (FR); Lizabeth-Anne Coffey-Dawe, Aulnay Sous Bois (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/592,172

(22) Filed: Nov. 3, 2006

Related U.S. Application Data
(60) Provisional application No. 60/734,300, filed on Nov. 8, 2005.

(30) Foreign Application Priority Data
Nov. 4, 2005 (FR) 05 53350

(51) Int. Cl. A61K 8/73; A61K 8/37

(52) U.S. Cl. 424/61

(57) ABSTRACT

The subject of the present invention is a nail varnish comprising a cellulose ester and an organic solvent medium comprising ethyl acetate and butyl acetate in an ethyl acetate/butyl acetate ratio ranging from 87/25 to 100/1. The subject of the present invention is also the use of such a varnish to obtain a film on the nails which dries rapidly, which is glossy, has good adherence and is water-resistant.

### CROSS REFERENCES TO RELATED APPLICATIONS

The present application claims priority from French application no. 05 53350 and U.S. provisional application No. 60/734,300, the entire contents of both of which are hereby incorporated by reference.

### FIELD OF THE INVENTION

The present invention relates to a nail varnish comprising an organic solvent medium comprising at least two solvents in a specific ratio and a cellulose ester.

The coloured or transparent nail varnish composition may be used as a varnish base or “base coat”, as a makeup product for the nails, as a finishing composition, also called “top coat”, to be applied over the makeup product for the nails or alternatively as a cosmetic care product for the nails.

### BACKGROUND OF THE INVENTION

In general, nail varnishes comprise at least one film-forming agent, one plasticizing agent, one gelling agent and one solvent medium which may be aqueous or organic.

The solvent may be an alcohol such as ethanol, isopropanol or butanol, an acetate such as ethyl or butyl acetate, a ketone.

Their use consists in applying one or more layers of the said varnish over the surface of the nail, and then in evaporating the solvent so as to deposit a film on the nail.

The layers of nail varnish deposited exhibit however, immediately after their application, risks of damage by rubbing and/or sticking to foreign bodies. Also, it is desirable that these varnishes dry rapidly after they have been applied.

In the state of the art, the rate of drying is optimized by selecting the volatile components, that is to say the solvent and optionally the diluent, entering into the composition of the nail varnish, according to their chemical nature and their volatility.

This optimization is constrained by a limitation of the nail varnish formulation because the components that are too volatile can penalize the quality of the makeup obtained. Indeed, defects may appear during the application of the nail varnish: formation of streaks on the nail varnish, excessively rapid drying of the brush during application. A non-homogeneous film is obtained which is not sufficiently smooth; it is not very glossy and it has an unsatisfactory adherence.

### SUMMARY OF THE INVENTION

The applicant has observed that the use of a cellulose ester and ethyl acetate in a predominant proportion in the solvent medium of a nail varnish makes it possible to obtain, after application to the nails, a film which dries rapidly while being homogeneous and smooth; and exhibiting good adherence and gloss properties.

The subject of the present invention is therefore a nail varnish comprising a cellulose ester and an organic solvent medium comprising ethyl acetate and butyl acetate in an ethyl acetate/butyl acetate ratio ranging from 87/25 to 100/1, preferably from 88/24 to 10/1, and even better from 88/24 to 6/1, the ethyl acetate being present in an amount greater than 25% by weight relative to the total weight of the composition and said composition comprising less than 5% by weight, relative to the total weight of the composition, of acetone.

The subject of the invention is also a cosmetic method for making up or for non-therapeutic care of the nails, comprising the application, to the nails, of at least one layer of a nail varnish composition as defined above.

The subject of the invention is also the use of a nail varnish composition comprising at least one cellulose ester and one organic solvent medium comprising ethyl acetate and butyl acetate in an ethyl acetate/butyl acetate ratio ranging from 87/25 to 100/1, the ethyl acetate being present in an amount greater than 25% by weight relative to the total weight of the composition and said composition comprising less than 5% by weight, relative to the total weight of the composition, of acetone, to obtain, after deposition on the nail, a film which dries rapidly, which is glossy, has good adherence and is water-resistant.

### DETAILED DESCRIPTION OF THE INVENTION

The nail varnish comprises a cosmetically acceptable medium, that is to say a medium that is non-toxic and capable of being applied to the keratin materials of human beings, in particular the nails.

Organic Solvent Medium

The organic solvent medium of the cosmetic composition comprises ethyl acetate and butyl acetate in an ethyl acetate/butyl acetate ratio ranging from 87/25 to 100/1, preferably from 88/24 to 10/1, and even better from 88/24 to 6/1.

Ethyl acetate may be present in an amount ranging from 25 to 70% by weight relative to the total weight of the composition, preferably from 30 to 60% by weight, and better from 35 to 50% by weight.

Ethyl acetate is present in an amount greater than 25% by weight relative to the total weight of the composition, preferably greater than 30% by weight and even better greater than 35% by weight.

Butyl acetate may be present in an amount ranging from 1 to 30% by weight relative to the total weight of the composition, preferably from 5 to 20% by weight.

According to one embodiment, the composition comprises butyl acetate in an amount of less than or equal to 30% by weight relative to the total weight of the composition, preferably less than or equal to 25% by weight, more preferably still less than or equal to 20% by weight, and even better less than or equal to 15% by weight.

The organic solvent medium of the composition may comprise, in addition to ethyl acetate and butyl acetate, a so-called additional organic solvent or a mixture of additional organic solvents.

The additional organic solvent may be chosen from:

ketones that are liquid at room temperature, such as methyl ethyl ketone, methyl isobutyl ketone, diisobutyl ketone, isophorone, cyclohexanone, acetone;

alcohols that are liquid at room temperature, such as ethanol, isopropanol, diacetone alcohol, 2-butoxyethanol, cyclohexanol;

propylene glycol ethers that are liquid at room temperature, such as propylene glycol monomethyl ether, propylene glycol monomethyl ether acetate, dipropylene glycol mono-n-butyl ether;

cyclic ethers such as γ-butyrolactone;

short-chain esters (having from 3 to 8 carbon atoms in total) other than ethyl acetate and butyl acetate, such as methyl acetate, propyl acetate, isopropyl acetate, isopentyl acetate, methoxypropyl acetate, butyl acetate;

ethers that are liquid at room temperature, such as diethyl ether, dimethyl ether or dichlorodiethyl ether;

alkanes that are liquid at room temperature, such as decane, heptane, dodecane, cyclohexane;

alkyl sulphoxides such as dimethyl sulphoxide;

aldehydes that are liquid at room temperature, such as benzaldehyde, acetaldehyde;

ethyl 3-ethoxypropionate;

carbonates such as propylene carbonate, dimethyl carbonate;

acetals such s methylal;

and mixtures thereof.

Preferably, ethyl acetate and butyl acetate are the predominant solvents by weight relative to the total weight of the organic solvents of the organic solvent medium of the composition.

The nail varnish according to the invention comprises less than 5% by weight, relative to the total weight of the composition, of acetone, preferably less than 3% and even better is completely free of acetone.

The organic solvent medium may represent from 10 to 95% by weight, relative to the total weight of the composition, preferably from 15% to 80% by weight, and even better from 20 to 70% by weight.

The cosmetically acceptable medium of the composition according to the invention may optionally comprise an aqueous medium.

The content of the aqueous medium of the composition may range from 5 to 95% by weight relative to the total weight of the composition, preferably from 50 to 70% by weight.

Film-Forming Polymer

The composition advantageously comprises a film-forming polymer chosen from cellulose esters.

According to the present invention, “film-forming polymer” is understood to mean a polymer capable of forming, on its own or in the presence of a film-forming aid, a continuous and adherent film on a support, in particular on keratin materials.

The cellulose esters may be chosen from esters obtained from the reaction of part of the free hydroxyl functional groups of cellulose with a carboxylic acid or a carboxylic acid derivative comprising from 1 to 10 carbon atoms, such as cellulose acetates, propionates, butyrates, isobutyrates, acetobutyrates, acetopropionates and mixtures thereof.

The cellulose ester may be present in the composition according to the invention as a dry matter content ranging from 0.1% to 20% by weight, relative to the total weight of the composition, preferably ranging from 0.5% to 15% by weight, and even better from 1% to 10% by weight.

The composition advantageously comprises at least one additional film-forming polymer chosen from synthetic polymers, of the free-radical type or polycondensate type, polymers of natural origin and mixtures thereof.

The additional film-forming polymer may be chosen in particular from cellulose polymers such as nitrocellulose, ethyl cellulose, or alternatively polyurethanes, acrylic polymers, vinyl polymers, polyvinylbutyrals, alkyd resins, resins obtained from the products of condensation of aldehyde, such as arylsulphonamide-formaldehyde resins such as toluenesulphonamide-formaldehyde resin, arylsulphonamide epoxy resins or alternatively ethyl tosylamide resins.

As additional film-forming polymer, use may be made in particular of nitrocellulose RS ⅛ sec.; RS ¼ sec.; RS ½ sec.; RS 5 sec.; RS 15 sec.; RS 35 sec.; RS 75 sec.; RS 150 sec.; AS ¼ sec.; AS ½ sec.; SS ¼ sec.; SS ½ sec.; SS 5 sec., marketed in particular by the company HERCULES; the toluenesulphonamide-formaldehyde resins “Ketjentflex MS80” from the company AKZO or “Santolite MHP”, “Santolite MS 80” from the company FACONNIER or “RESIMPOL 80” from the company PAN AMERICANA, the alkyd resin “BECKOSOL ODE 230-70-E” from the company DAINIPPON, the acrylic resin “ACRYLOID B66” from the company ROHM & HAAS, the polyurethane resin “TRIXENE PR 4127” from the company BAXENDEN.

According to one embodiment of the invention, the film-forming polymer is a film-forming linear block ethylene polymer which preferably comprises at least a first block and at least a second block having different glass transition temperatures (Tg), the said first and second blocks being linked to each other by an intermediate block comprising at least one constituent monomer of the first block and at least one constituent monomer of the second block. Advantageously, the first and second blocks of the block polymer are incompatible with each other.

Such polymers are described for example in the documents EP 1411069 or WO 04/028488.

The film-forming polymer may be present in the composition according to the invention in a dry matter content ranging from 0.1% to 60% by weight, relative to the total weight of the composition, preferably ranging from 2% to 40% by weight, and even better from 5% to 25% by weight.

Film-Forming Aid

To improve the film-forming properties of the nail varnish composition, a film-forming aid may be provided.

Such a film-forming aid may be chosen from any of the compounds known to a person skilled in the art as being capable of fulfilling the desired function, and may be chosen in particular from plasticizing agents and agents for coalescing the film-forming polymer(s).

Thus, the composition may additionally comprise at least one plasticizing agent and/or one coalescing agent. In particular, there may be mentioned, alone or as a mixture, the customary plasticizers and coalescing agents, such as:

glycols and derivatives thereof, such as diethylene glycol ethyl ether, diethylene glycol methyl ether, diethylene glycol butyl ether or alternatively diethylene glycol hexyl ether, ethylene glycol ethyl ether, ethylene glycol butyl ether, ethylene glycol hexyl ether;

glycol esters;

propylene glycol derivatives, and in particular propylene glycol phenyl ether, propylene glycol diacetate, dipropylene glycol ethyl ether, tripropylene glycol methyl ether and diethylene glycol methyl ether, propylene glycol butyl ether;

acid, in particular carboxylic acid, esters such as citrates, phthalates, adipates, carbonates, tartrates, phosphates, sebacates;

oxyethylenated derivatives, such as oxyethylenated oils, in particular vegetable oils, such as castor oil; and

mixtures thereof.

The type and quantity of plasticizing and/or coalescing agent may be chosen by persons skilled in the art on the basis of their general knowledge.

For example, the content of plasticizing and/or coalescing agent may range from 0.01% to 20%, and in particular from 0.5% to 10% by weight relative to the total weight of the composition.

Thickening agent

The composition may comprise a thickening agent which may be chosen in particular from: hydrophobic silicas such as those described in the document EP-A-898960, for example marketed under the references “AEROSIL R812®” by the company Degussa, “CAB-O-SIL TS-530®”, “CAB-O-SIL TS-610®”, “CAB-O-SIL TS-720®” by the company Cabot, “AEROSIL R972®”, “AEROSIL R974®” by the company Degussa; hydrophilic silicas such as that marketed under the reference “AEROSIL 200®” by the company Degussa, clays such as montmorillonite, modified clays such as bentones for example, stearalkonium hectorite, stearalkonium bentonite, alkyl ethers of polysaccharides (in particular whose alkyl group comprises from 1 to 24 carbon atoms, preferably from 1 to 10, even better from 1 to 6, and more especially from 1 to 3) such as those described in the document EP-A-898958.

The total proportion of thickening agent(s) in the compositions according to the invention may range from 0.01 to 15% by weight, relative to the total weight of the composition, preferably from 0.5 to 15%, and even better from 0.5 to 10% by weight.

Colouring Material

The composition according to the invention may additionally comprise one or more colouring materials chosen from water-soluble colorants, and pulverulent colouring materials, such as pigments, pearlescent agents and glitter which are well known to a person skilled in the art. The colouring materials may be present in the composition in an amount ranging from 0.01% to 50% by weight, relative to the weight of the composition, preferably from 0.01% to 30% by weight.

The expression pigments should be understood to mean white or coloured, inorganic or organic particles of any shape which are insoluble in the physiological medium, intended to colour the composition.

The expression pearlescent agents should be understood to mean iridescent particles of any shape, in particular produced by certain molluscs in their shell or else synthesized.

The pigments may be white or coloured, inorganic and/or organic. There may be mentioned, among the inorganic pigments, titanium dioxide, optionally surface-treated, zirconium or cerium oxides, and zinc oxide, iron oxide (black, yellow or red) or chromium oxide, manganese violet, ultramarine blue, chromium hydrate and ferric blue, metallic powders such as aluminium powder or copper powder.

Among the organic pigments, there may be mentioned carbon black, D & C type pigments, and lacquers based on carmine, barium, strontium, calcium, aluminium.

The pearlescent pigments may be chosen from white pearlescent pigments such as mica coated with titanium or with bismuth oxychloride, coloured pearlescent pigments such as mica-titanium coated with iron oxides, mica-titanium coated with in particular ferric blue or chromium oxide, mica-titanium coated with an organic pigment of the abovementioned type and pearlescent pigments based on bismuth oxychloride.

The water-soluble colorants are for example beet juice, methylene blue.

The composition according to the invention may additionally comprise one or more fillers, in particular in an amount ranging from 0.01% to 50% by weight, relative to the total weight of the composition, preferably ranging from 0.01% to 30% by weight. The expression fillers should be understood to mean colourless or white, inorganic or synthetic particles of any shape which are insoluble in the medium of the composition regardless of the temperature at which the composition is manufactured. These fillers serve in particular to modify the rheology or the texture of the composition.

The fillers may be inorganic or organic, of any shape, platelets, spherical or oblong, regardless of the crystallographic shape (for example sheet, cubic, hexagonal, orthorhombic and the like). There may be mentioned talc, mica, silica, kaolin, polyamide powder (Nylon®) (Orgasol® from Atochem), poly-β-alanine powder and polyethylene powder, tetrafluoroethylene polymer powders (Teflon®), lauroyllysine, starch, boron nitride, polymeric hollow microspheres such as those of polyvinylidene chloride/acrylonitrile such as Expancel® (Nobel Industrie), acrylic acid copolymers (Polytrap® from the company Dow Corning) and silicone resin microbeads (Tospearls® from Toshiba, for example), polyorganosiloxane elastomer particles, precipitated calcium carbonate, magnesium carbonate and hydrocarbonate, hydroxyapatite, hollow silica microspheres (Silica Beads® from Maprecos), glass or ceramic microcapsules, metal soaps derived from organic carboxylic acids having from 8 to 22 carbon atoms, preferably from 12 to 18 carbon atoms, for example zinc, magnesium or lithium stearate, zinc laurate, magnesium myristate.

Other Additives

The composition may additionally comprise other ingredients commonly used in cosmetic compositions. Such ingredients may be chosen from spreading agents, wetting agents, dispersing agents, antifoams, preservatives, UV-screening agents, active agents, surfactants, moisturizing agents, perfumes, neutralizing agents, stabilizers and antioxidants.

Of course, persons skilled in the art will be careful to choose this or these optional additional compounds, and/or their quantity, such that the advantageous properties of the composition for use according to the invention are not, or not substantially, impaired by the addition envisaged.

According to another aspect, the subject of the invention is a nail varnish product comprising: i) a container delimiting at least one compartment, the said container being closed by a closing element, and ii) a composition according to the invention which is placed inside the said compartment.

The container may be of any suitable form. It may be in particular in the form of a bottle and may, at least in part, be made of a material such as glass. However, materials other than glass may be used, such as thermoplastic materials such as PP or PE or such as a metal.

The closing element may be coupled to the compartment by screwing in the closed position of the container. Alternatively, the coupling between the closing element and the container may be made other than by screwing, in particular by snap fastening.

The container is preferably equipped with an applicator which may be in the form of a brush consisting of at least one tuft of bristles. Alternatively, the applicator is provided in a form other than a brush, for example in the form of a spatula or a foam tip.

The examples which follow illustrate the invention without limitation. Unless otherwise stated, the quantities indicated are percentages by mass.

The invention is illustrated in greater detail in the following examples. Unless otherwise stated, the quantities are given as a percentage by weight relative to the total weight of the composition.

### EXAMPLES 1 TO 5

### Nail Varnishes

Nail varnishes having the following composition (% by weight) are prepared:

| | Example | Example | Example | Example | Example |
|---|---|---|---|---|---|
| | 1 | 2 | 3 | 4 | 5 |
| Cellulose acetobutyrate | 2.976 | 2.976 | 2.976 | 2.976 | 2.976 |
| (CAB 381.05 from Eastman | | | | | |
| Chemical) | | | | | |
| Nitrocellulose containing | 10.35 | 10.44 | 10.6 | 8.84 | 9.54 |
| 30% of isopropyl alcohol | | | | | |
| (viscosity: E22 - ½ S) | | | | | |
| Nitrocellulose containing | 1.68 | 1.64 | 1.7 | 1.92 | 2.84 |
| 30% of isopropyl alcohol | | | | | |
| (Azur E80 from Bergerac) | | | | | |
| N-ethyl-O,P-toluene- | 4.09 | 3.91 | 4 | 4.25 | 4.75 |
| sulphonamide resin | | | | | |
| Glycerophthalic alkyd | 13.40 | 13.47 | 13.5 | 11.9 | 13.56 |
| resin esterified with | | | | | |
| branched fatty acids⁽¹⁾ | | | | | |
| in ethyl acetate at 70% | | | | | |
| Isopropyl alcohol | 3.1 | 3.11 | 3.13 | 2.52 | 3.23 |
| Stearylbenzyldimethyl- | 0.98 | 0.99 | 1 | 0.95 | — |
| ammonium-modified | | | | | |
| hectorite (BENTONE | | | | | |
| 27 V from Elementis) | | | | | |
| Red 7 lacquer | 0.0075 | 0.75 | 0.08 | — | — |
| Yellow 5 lacquer | 0.01 | — | — | — | — |
| Red 6 lacquer | 0.012 | — | — | — | — |
| Polyethylene oxide- | 1.25 | 0.16 | 0.05 | — | — |
| treated titanium oxide | | | | | |
| Mica-titanium oxide | | | | 10 | — |
| Black iron oxide | 0.002 | — | — | — | — |
| Red 34 lacquer | — | — | 0.318 | — | — |
| Ethyl acetate | 47.06 | 46.34 | 46.9 | 45 | qs 100 |
| Tributyl acetylcitrate | 2.71 | 2.92 | 2.9 | 1.94 | 2.11 |
| Butyl acetate | 12.31 | 13.22 | 12.9 | 9.6 | — |
| Citric acid, 1 H2O | 0.98 | 0.99 | 0.04 | 0.04 | — |

⁽¹⁾marketed by SHELL under the trade name CARDURA 30 ®.

### EXAMPLES 6 TO 8

### Nail Varnishes

A nail varnish according to the invention (Examples 6 and 7) is prepared which comprises a cellulose ester (cellulose acetobutyrate) and ethyl acetate and butyl acetate as ethyl acetate/butyl acetate of about 3.9 and a varnish not in accordance with the invention (Example 8) which does not comprise a cellulose ester.

| | Example | Example | Example |
|---|---|---|---|
| | 6 | 7 | 8 |
| Cellulose acetobutyrate (CAB | 8.17 | 4.08 | — |
| 381.05 from Eastman Chemical) | | | |
| Nitrocellulose containing 30% | 2.9 | 7.64 | 12.38 |
| of isopropyl alcohol (viscosity: | | | |
| E22 - ½ S) | | | |
| Nitrocellulose containing 30% of | — | 1.09 | 2.18 |
| isopropyl alcohol (Azur E80 from | | | |
| Bergerac) | | | |
| N-ethyl-O,P-toluenesulphon- | 5.07 | 5.07 | 5.07 |
| amide resin | | | |
| Glycerophthalic alkyd resin | 16.14 | 16.14 | 16.14 |
| esterified with branched fatty | | | |
| acids⁽¹⁾ in ethyl acetate at 70% | | | |
| Isopropyl alcohol | 7.25 | 5.5 | 3.75 |
| Stearylbenzyldimethylammonium- | 1.15 | 1.15 | |
| modified hectorite (BENTONE 27 | | | |
| V from Elementis) | | | |
| Ethyl acetate | 45.78 | 45.78 | 45.78 |
| Tributyl acetylcitrate | 1.78 | 1.78 | 1.78 |
| Butyl acetate | 11.68 | 11.68 | 11.68 |
| Citric acid, 1 H2O | 0.04 | 0.04 | 0.04 |

⁽¹⁾marketed by SHELL under the trade name CARDURA 30 ®.

The varnishes of Examples 6 and 7 (according to the invention), after application to the nails, were judged to dry more rapidly than that of Example 8 (not in accordance with the invention).

1. Nail varnish comprising a cellulose ester and an organic solvent medium comprising ethyl acetate and butyl acetate in an ethyl acetate/butyl acetate ratio ranging from 87/25 to 100/1, the ethyl acetate being present in an amount greater than 25% by weight relative to the total weight of the composition and said composition comprising less than 5% by weight, relative to the total weight of the composition, of acetone.

2. The nail varnish according to claim 1, wherein the ethyl acetate/butyl acetate ratio ranges from 88/24 to 10/1.

3. The nail varnish according to claim 1, wherein the ethyl acetate is present in an amount ranging from 25 to 70% by weight relative to the total weight of the composition.

4. The nail varnish according to claim 1, wherein the ethyl acetate is present in an amount greater than 30% by weight relative to the total weight of the composition.

5. The nail varnish according to claim 1, wherein the butyl acetate is present in an amount ranging from 1 to 30% by weight relative to the total weight of the composition.

6. The nail varnish according to claim 1, wherein the butyl acetate is present in an amount of less than or equal to 30% by weight relative to the total weight of the composition.

7. The nail varnish according to claim 1, wherein the cellulose ester is an ester obtained from the reaction of part of the free hydroxyl functional groups of cellulose with a carboxylic acid or a carboxylic acid derivative comprising from 1 to 4 carbon atoms.

8. The nail varnish according to claim 1, wherein the cellulose ester is a cellulose acetobutyrate.

9. The nail varnish according to claim 1, wherein the cellulose ester represents from 0.1% to 20% by weight, relative to the total weight of the composition.

10. The nail varnish according to claim 1, further comprising at least one additional organic solvent.

11. The nail varnish according to claim 1, wherein the ethyl acetate and the butyl acetate are the predominant solvents by weight relative to the total weight of the organic solvents of the organic solvent medium of the composition.

12. The nail varnish according to claim 1, further comprising an aqueous medium.

13. The nail varnish according to claim 1, further comprising at least one additional film-forming polymer.

14. The nail varnish according to claim 13, wherein the additional film-forming polymer is selected from the group consisting of cellulose polymers, polyurethanes, acrylic polymers, vinyl polymers, polyvinylbutyrals, alkyd resins, resins obtained from the products of condensation of aldehyde, arylsulphonamide epoxy resins, and ethyl tosylamide resins.

15. The nail varnish according to claim 13, the additional film-forming polymer is present in an amount ranging from 0.1% to 60% by weight, relative to the total weight of the composition.

16. The nail varnish according to claim 1, further comprising at least one film-forming aid and/or one thickening agent.

17. The nail varnish according to claim 1, further comprising at least one colouring material.

18. The nail varnish according to claim 17, wherein the colouring material is present in an amount ranging from 0.01% to 50% by weight, relative to the weight of the composition.

19. A cosmetic method for making up or for non-therapeutic care of the nails, comprising applying to the nails at least one layer of a nail varnish composition according to claim 1.

20. A method of forming a film on a nail comprising applying to the nail a nail varnish composition comprising at least one cellulose ester and one organic solvent medium comprising ethyl acetate and butyl acetate in an ethyl acetate/butyl acetate ratio ranging from 87/25 to 100/1, the ethyl acetate being present in an amount greater than 25% by weight relative to the total weight of the composition and said composition comprising less than 5% by weight, relative to the total weight of the composition, of acetone, wherein after application of the composition to the nail, a film is formed which is glossy, has good adherence and is water-resistant.
